(19) United States
(12) Patent Application Publication
(10) Pub. No.: US 2025/0170229 A1
(43) Pub. Date: May 29, 2025

(54) ESTABLISHMENT AND TRANSLATION OF NEAR-ZERO IMMUNOSUPPRESSION STRATEGY FOR ORTHOTOPIC LIVER ALLOGRAFT TRANSPLANTATION

(71) Applicant: Wentao ZHOU, Xiaogan (CN)

(72) Inventor: Wentao ZHOU, Xiaogan (CN)

(21) Appl. No.: 18/519,115

(22) Filed: Nov. 27, 2023

(51) Int. Cl. A61K 39/00; A61K 38/13; A61K 38/19; A61P 37/06

(52) U.S. Cl. CPC A61K 39/001; A61K 38/13; A61K 38/193; A61P 37/06

(57) ABSTRACT

A construction method of an immune tolerance induction scheme for orthotopic liver transplantation and an animal model thereof are provided, a design of the animal model is clever, reasonable, and has good repeatability, and the animal model meets requirements of rat orthotopic liver transplantation and is similar to human orthotopic liver transplantation. A postoperative survival rate is high, repeatability is good, and a tolerance rate is as high as 100%, which are confirmed by liver function testing and pathological analysis after operation, and the immune tolerance induction scheme can effectively solve problems of long time, high mortality rate, and high requirements for personnel and microscopy involved in research objectives and complete replication process of liver transplantation. An application of tolerance induction that can be extended to different combinations of other donors and receptors simultaneously.

### TECHNICAL FIELD

The disclosure relates to the field of animal model construction technologies and tolerance induction schemes thereof, and more particularly to a construction method for an animal model of an orthotopic liver transplantation and a determination of an immune tolerance induction scheme.

### BACKGROUND

Liver transplantation (LT) is recognized as an only effective treatment for end-stage liver disease. However, a result of big data research reported that there has been almost no progress in long-term survival of liver transplantation recipients, mainly due to the long-time use of immunosuppressants, which brings a series of complications as follows. For example, renal insufficiency occurs in 25% of recipients; neurotoxicity occurs in 10-30% of recipients; diabetes occurs in 9-18% of recipients; cardiovascular disease occurs in 20-33% of kidney recipients, 26-40% of liver recipients and 29% of lung recipients; infection, the fourth cause of death in transplantation, occurs in ⅔ of recipients; de novo malignancy has an incidence of 11 times higher than that of normal subjects; digestive system diseases occur in 12% of recipients; hyperlipidemia occurs in 46% of recipients; and hematological diseases occur in 30% of recipients. In this situation, development and quality of life of the recipients are unsatisfactory, and economic costs of the recipients are huge, if a liver transplantation recipient survives for 15 years, it will cost 60-70,000 yuan per year, and totaling over 1 million yuan. A tolerance scheme implemented and applied in human being can not only greatly reduce the aforementioned complications, but also save a huge cost of 1 million yuan per person. The number of liver transplantation cases in China is 5000 per year, thus saving a total cost of 5 billion yuan per year. The number of the liver transplantation cases in the world is 20,000 per year, thus saving the total cost about 20 billion, one donor liver can satisfy two recipients, the number of transplantation recipients are doubled theoretically, thus saving the total cost about 40 billion.

Immune tolerance refers to chronic rejection (Banff standard) with good graft function and no pathological confirmation when the immunosuppressants are completely discontinued. The immune tolerance state maintains for at least 1 year for human organ transplantation, and the immune tolerance state maintains about 2 months for rats. Induced tolerance is known as the holy grail of transplantation immunology, the methods for the induced tolerance are summarized as follows: (1) clearance of T-cells; (2) blocking of co-stimulation pathways; (3) induction of mixed chimeras; and (4) improvement of regulatory T cells. However, the above methods involve intervention in an immune system of the recipient and have significant side effects. The existing methods focus on donor-specific immune unresponsiveness and sustained maintenance of the mixed chimeras.

At present, a general tolerance model adopts a whole-liver transplant, however, there is no clinical application scheme in the world.

Therefore, it is urgent to research an application scheme for researching immune tolerance of liver transplantation.

### SUMMARY

In order to solve the above problem, a new model and a new scheme of immune tolerance are constructed from transplants. Specifically, a construction method of immune tolerance induction for orthotopic liver transplantation includes the following steps.

A process of a donor operation includes the following steps (1)-(9).

In step (1), specific pathogen free (SPF) level rats (Sprague-Dawley abbreviated as SD, Lewis) weighing 200-350 grams (g) fed at a room temperature of 20-24 Celsius degrees (° C.) and humidity of 40-60% are selected and fasted for 12 hours (h) before the operation, but not forbidden water.

In step (2), rats with a weight difference of no more than 30 g are selected as donors, each donor rat is anesthetized by inhaling isoflurane, and abdomen of the donor rat is shaved and disinfected.

In step (3), the donor rat is fixed on an operating table in a supine position, and is kept under anesthesia by continuously inhaling the isoflurane, abdominal cavity of the donor rat is opened through a transverse incision, and a liver of the donor rat is exposed by pulling xiphoid process.

In step (4), intestinal canal of the donor rat is pulled out of a left side of the abdominal cavity and is wrapped in a wet gauze, and abdominal aorta below a horizontal of right renal vein is dissociated.

In step (5), the abdominal aorta on celiac trunk is exposed and blocked with a vascular clamp.

In step (6), 5 milliliters (mL) of physiological saline containing low molecular weight heparin (LMWH) with a concentration of 625 international units per kilogram (IU/kg) are injected into the abdominal aorta below the horizontal of the right renal vein with a No. 1 needle, to perform systemic heparinization on the donor rat, and retrograde perfusion is performed on the liver of the donor rat.

In step (7), an oblique incision is cut in superior mesenteric vein, and 10 mL of lactated Ringer's solution (also referred to as Ringer's lactate solution) is injected to preform reperfusion on the liver; inferior vena cava below the horizontal of renal veins is cut open to bleed until the donor rat dies, the anesthesia is stopped, and physiological saline at a temperature of 0-4° C. is poured into the liver for multiple times to prevent the liver rewarming.

In step (8), branches of the superior mesenteric vein and splenic vein are ligated, and the superior mesenteric vein is dissociated. The celiac trunk is dissociated, and splenic artery and left gastric artery are ligated. 5 millimeters (mm) of extrahepatic bile duct is retained, and a support tube is inserted into bile duct and fixed. Pyloric vein and gastroduodenal artery are ligated together. Left suprarenal vein and right suprarenal vein are dissociated and ligated, the right renal vein is ligated with a 10-0 silk thread (also referred to as 10-0 suture), and the inferior vena cava is cut off at the left renal vein horizontally.

In step (9), perihepatic tissues of the donor rat are separated counterclockwise, left inferior phrenic vein is ligated tightly against the liver, hepatic vein is cut off tightly against diaphragm, the liver of the donor rat is removed, and placed into lactated Ringer's solution at a temperature of 0-4° C. On the background, portal vein and the inferior vena cava are sleeved with sleeves respectively. When the transplant is a half-liver (50%) transplant, left lobe, caudate lobe and left half of middle lobe of the liver are ligated and removed; and when the transplant is a partial-liver (70%) transplant, the left lobe of liver is ligated and removed.

A process of a recipient operation includes the following steps (1)-(8).

In step (1), SPF level rats (Brown Norway abbreviated as BN, Lewis) weighing 200-350 g fed at the room temperature of 20-24° C. and the humidity of 40-60% are selected fasted for 12 h before the operation, but not forbidden water.

In step (2), rats with a weight difference larger than 30 g of the donor rats are selected as recipients, each recipient rat is anesthetized by inhaling the isoflurane, and abdomen of the recipient rat is shaved and disinfected.

In step (3), the recipient rat is fixed on the operating table in the supine position, and is kept under anesthesia by continuously inhaling the isoflurane, abdominal cavity of the recipient rat is opened through a longitudinal incision or a transverse incision, and a liver of the recipient rat is exposed by pulling xiphoid process.

In step (4), perihepatic tissues of the recipient rat are separated and cut off, and the liver of the recipient rat is removed; and the isoflurane is stopped inhaling.

In step (5), the liver of the donor rat is placed orthotopically, and supra-hepatic inferior vena cava (i.e., inferior vena cava on the liver) is sutured. The portal vein is connected through a cuff method (also referred to as sleeve anastomosis), and the inferior vena cava is connected through the cuff method to thereby restore blood flow, 10 mL of the physiological saline is injected through dorsal vein of penis to help restore blood circulation, a support tube method is used to restore hepatic artery blood flow, and the same method is used to connect bile duct.

In step (6), muscle layers of the incision are continuously sutured with a 5-0 silk thread, a lidocaine mortar is applied in the incision to relieve pain, and a skin layer is intermittently sutured. Cyclosporin (CSA) with a concentration of 5-8 milligrams per kilogram (mg/kg) is subcutaneously injected during the operation, and 1.2 mL of ceftizoxime sodium is intraperitoneally injected.

In step (7), the recipient rat is indirectly irradiated by an infrared lamp to keep a temperature for 12 h when the recipient rat is awake from anesthesia after the operation, and a survival of the recipient rat is observed.

In step (8), an experimental group and a control group 1 are subcutaneously injected granulocyte colony-stimulating factor (GSF) with a concentration of 150 micrograms per kilogram (μg/kg) after completing the operation about 4 h, and then the experimental group and the control group 1 are subcutaneously injected GSF with a concentration of 50-150 μg/kg once a day, and a total of 5 times; the experimental group and the control group 1 are subcutaneously injected the ceftizoxime sodium with a concentration of 100 mg/kg once a day, and a total of 3 times; and the experimental group and the control group 1 are injected CSA with a concentration of 3-4 mg/kg once a day, and a total of 9 times. A control group 2 is not injected GSF, and others are the same.

Activity, drinking water, urine volume, urine color and wound of the recipient rat are observed daily.

After adopting the above method, the disclosure has the following advantages. A design of the disclosure is reasonable and clever, the half-liver (50%) transplant model is widely adopted in clinical practice. The disclosure has a simple operation and a good repeatability, which can simulate the clinical practice well, and accord with characteristics of immune tolerance after the orthotopic liver transplantation in the rats. The function of the liver transplant is normal, the transplant does not have the acute or chronic rejection reactions, a structure of the transplant is normal, survival rate of the recipients is high, and a tolerance rate reaches 100%. It can effectively solve problems of long time, high mortality and high technical requirements of personnel involved during a process of completely replicating liver transplantation, and effectively solve problems of effectiveness, simplicity, repeatability and operability of the tolerance scheme.

### BRIEF DESCRIPTION OF DRAWINGS

FIGS. 1A-1C illustrate schematic diagrams of a whole-liver transplant and the whole-liver transplant after transplantation of a control group 1 of the disclosure.

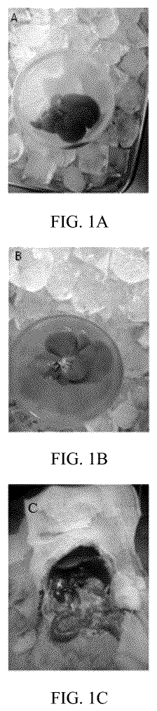

FIGS. 1D-1F illustrate schematic diagrams of a half-liver transplant and the half-liver transplant after transplantation of a control group 2 and a tolerance group of the disclosure.

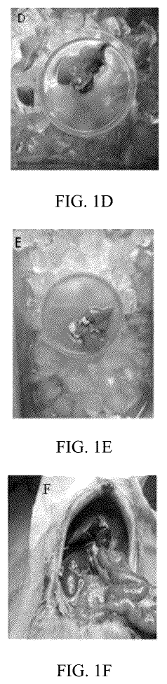

FIG. 2 illustrates a schematic diagram of survival of immune tolerant rats after an orthotopic liver transplantation in rats of the disclosure. A chronic rejection or related death of a transplant, confirmed by pathology, is recorded as a survival endpoint, and a time from transplantation to the survival endpoint is a survival time.

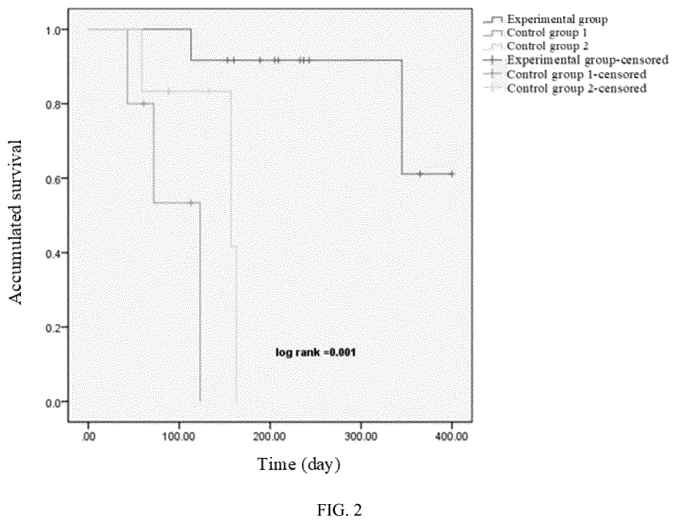

FIGS. 3A-3B illustrate schematic diagrams of liver function of rats in three groups after the orthotopic liver transplantation in the rats of the disclosure.

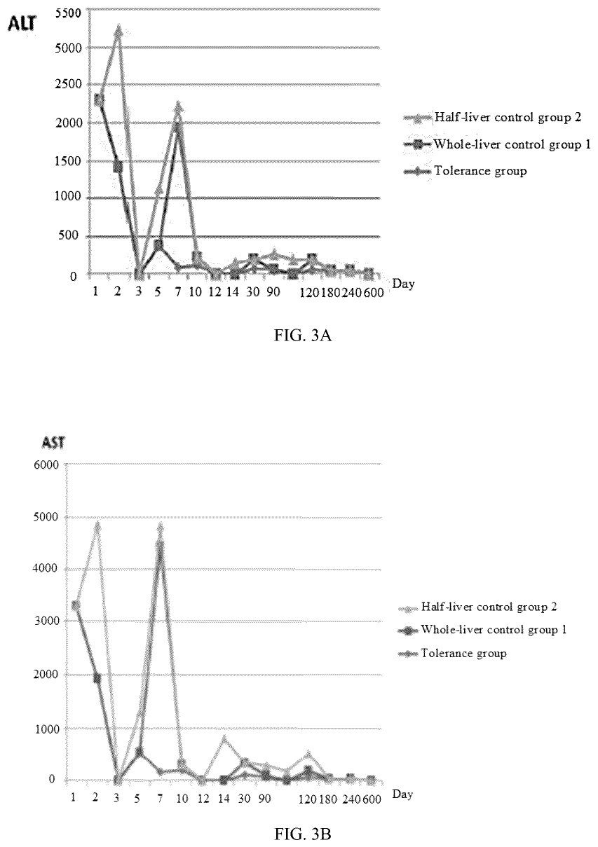

FIG. 4 illustrates a hematoxylin-eosin (HE) staining comparison diagram of immune tolerance transplants after the orthotopic liver transplantation in the rats of the disclosure.

### DETAILED DESCRIPTION OF EMBODIMENTS

Embodiment 1 is a process of a liver transplantation.

A process of a donor operation includes the following steps (1)-(9).

In step (1), specific pathogen free (SPF) level rats (Sprague-Dawley abbreviated as SD, Lewis) weighing 200-350 grams (g) fed at a room temperature of 20-24 Celsius degrees (° C.) and humidity of 40-60% are selected and fasted for 12 hours (h) before the operation, but not forbidden water.

In step (2), rats with a weight difference of no more than 30 g are selected as donors, each donor rat is anesthetized by inhaling isoflurane, and abdomen of the donor rat is shaved and disinfected.

In step (3), the donor rat is fixed on an operating table in a supine position, and is kept under anesthesia by continuously inhaling the isoflurane, abdominal cavity of the donor rat is opened through a transverse incision, and a liver of the donor rat is exposed by pulling xiphoid process.

In step (4), intestinal canal of the donor rat is pulled out of a left side of the abdominal cavity and is wrapped in a wet gauze, and abdominal aorta below a horizontal of right renal vein is dissociated.

In step (5), the abdominal aorta on celiac trunk is exposed and blocked with a vascular clamp.

In step (6), 5 milliliters (mL) of physiological saline containing low molecular weight heparin (LMWH) with a concentration of 625 international units per kilogram (IU/kg) are injected into the abdominal aorta below the horizontal of the right renal vein with a NO. 1 needle, to perform a systemic heparinization on the donor rat, and retrograde perfusion is performed on the liver of the donor rat.

In step (7), an oblique incision is cut in superior mesenteric vein, and 10 mL of lactated Ringer's solution (also refers to as Ringer's lactate solution) is injected to perform reperfusion on the liver; inferior vena cava below the horizontal of renal veins is cut open to bleed until the donor rat dies, the anesthesia is stopped, and physiological saline at a temperature of 0-4° C. is poured into the liver for multiple times to prevent the liver rewarming.

In step (8), branches of the superior mesenteric vein and splenic vein are ligated, and the superior mesenteric vein is dissociated. The celiac trunk is dissociated, and splenic artery and left gastric artery are ligated. 5 millimeters (mm) of extrahepatic bile duct is retained, and a support tube is inserted into bile duct and fixed. Pyloric vein and gastroduodenal artery are ligated together. Left suprarenal vein and right suprarenal vein are dissociated and ligated, the right renal vein is ligated with a 10-1 silk thread (also referred to as 10-0 suture), and the inferior vena cava is cut off at the left renal vein horizontally.

In step (9), perihepatic tissues of the donor rat are separated counterclockwise, left inferior phrenic vein is ligated tightly against the liver, hepatic vein is cut off tightly against diaphragm, the liver of the donor rat is removed, and placed into lactated Ringer's solution at a temperature of 0-4° C. On the background, portal vein and the inferior vena cava are sleeved with sleeves respectively. When the transplant is a half-liver (50%) transplant, left lobe, caudate lobe and left half of middle lobe of the liver are ligated and removed.

TABLE 1
Groups, process, and results of research

| Rat groups | Transplants | Numbers | Process | Survival time (day) |
|---|---|---|---|---|
| Lewis→BN^{#} (control | 100% | 14 | CSA + GSF | 45, 19, 61, 123, 43, 113, 38, 58, |
| group 1) | | | | 56, 29, 47, 24, 59, 60 |
| Lewis→BN^{#} (control | 50% | 13 | CSA | 163, 190, 133, 20, 157, 61, 59, 55, |
| group 2) | | | | 54, 69, 58, 60, 47 |
| Lewis→BN | 50% | 18 | CSA + GSF | 153, 160, 157, 189, 243, 237, 133, |
| (experimental group) | | | | 345, 132, 365, 508, 113, 420, 393, |
| | | | | 389, 233, 203, 194 |

Note:
Cyclosporin: CSA;
granulocyte colony-stimulating factor: GSF

A process of a recipient operation includes the following steps (1)-(8).

In step (1), SPF level rats (Brown Norway abbreviated as BN, Lewis) weighing 200-350 g fed at the room temperature of 20-24° C. and the humidity of 40-60% are selected and fasted for 12 h before the operation, but not forbidden water.

In step (2), rats with a weight difference of no more than 30 g are selected as recipients, each recipient rat is anesthetized by inhaling the isoflurane, and abdomen of the recipient rat is shaved and disinfected.

In step (3), the recipient rat is fixed on the operating table in the supine position, and is kept under anesthesia by continuously inhaling the isoflurane, abdominal cavity of the recipient rat is opened through a middle longitudinal incision, and a liver of the recipient rat is exposed by pulling xiphoid process; and left and right rib arches (also referred to as arcus costali) are pulled with self-made hooks using paper clips.

In step (4), perihepatic tissues of the recipient rat are separated and cut off counterclockwise from hepatogastric ligament, left inferior phrenic vein is ligated near diaphragm, left and right suprarenal veins are ligated, inferior vena cava is skeletonized to a horizontal of right renal vein, proper hepatic artery is ligated and cut off at a proximal end of common bile duct, diaphragm ring is clamped by using a mosquito clamp, portal vein and the inferior vena cava are blocked by using the vascular clamps, respectively, supra-hepatic inferior vena cava (i.e., inferior vena cava on the liver), infra-hepatic inferior vena cava (i.e., inferior vena cava below the liver), and the portal vein are cut off near the liver, and the liver of the recipient rat is removed; and the isoflurane is stopped inhaling.

In step (5), the liver of the donor rat is placed orthotopically, a posterior wall and an anterior wall of the supra-hepatic inferior vena cava are sutured by using an 8-0 silk thread (also referred to as 8-0 suture) from left to right with a one-point method, the mosquito clamp is removed, and the supra-hepatic inferior vena cava is blocked by using a concavo-convex tooth hemostatic clip. The portal vein is connected through a cuff method (also referred to as sleeve anastomosis), and the vascular clamp in the portal vein and the mosquito clamp in the supra-hepatic inferior vena cava are removed to restore portal vein blood flow. The inferior vena cava is connected through the cuff method to restore blood flow, 10 mL of the physiological saline is injected through dorsal vein of penis to help restore blood circulation. On a right side of the portal vein, common hepatic artery is blocked by using the vascular clamp, gastroduodenal artery is ligated, an oblique incision is cut on the common hepatic artery by using a scissors, a supporting tube of the artery of the liver of the donor rat is inserted into the common hepatic artery, and the supporting tube is ligated and fixed, and then opened to restore hepatic artery blood flow, and the same method is used to connect bile duct.

In step (6), a median incision is continuously sutured with a 4-0 silk thread, a lidocaine mortar is applied in the median incision to relieve pain, and a skin layer is intermittently sutured.

In step (7), the recipient rat is indirectly irradiated by an infrared lamp to keep a temperature for 12 h when the recipient rat is awake from anesthesia after the operation, the recipient rat is continued to be fed at the room temperature of 20-24° C. and the humidity of 40-60%, and a survival of the recipient rat is observed.

In step (8), the experimental group is subcutaneously injected GSF with a concentration of 50-150 micrograms per kilogram (μg/kg) after the operation, once a day, and a total of 5 times; the experimental group is subcutaneously injected ceftizoxime sodium with a concentration of 100 milligrams per kilogram (mg/kg) once a day, and a total of 3 times; and the experimental group is injected CSA with a concentration of 4 mg/kg once a day, and a total of 9 times. The control group 1 and control group 2 are subcutaneously injected the ceftizoxime sodium with a concentration of 100 mg/kg once a day, and a total of 3 times; and the control group 1 and control group 2 are injected CSA with a concentration of 4 mg/kg once a day, and a total of 9 times (e.g., Table 1).

In step (9), activity, drinking water, urine volume, urine color and wound of the recipient rat are observed every day.

### Embodiment 2

1. Experimental materials are transplanted rats after liver transplantation, the isoflurane, and a set of surgical instruments.

2. A preparation method includes the following steps (1)-(7).

In step (1), the transplanted rats fed at a room temperature of 23-27° C. and the humidity of 40-60% are selected.

In step (2), the recipient rats are weighed, and the recipient rats are divided into the control group 1 (including 14 recipient rats), the control group 2 (including 13 recipient rats) and the experimental group (including 18 recipient rats), and each recipient rat is anesthetized by inhaling the isoflurane, and abdomen of the recipient rat is shaved and disinfected.

In step (3), abdominal cavity of the recipient rat is opened through a median incision, and abdominal aorta of the recipient rat is exposed by pulling intestinal canal outward to the left, a blood taking needle is inserted into the abdominal aorta and is connected to a test tube containing ethylenediamine tetraacetic acid (EDTA) anticoagulant to obtain plasma, the test tube is inverted several times to prevent blood clotting.

In step (4), a speed of a centrifuge is adjusted to 2,000 revolutions per minute and centrifuged for 10 minutes (min) to obtain serum.

In step (5), the serum is transferred to a cryopreservation tube and labeled as a serum specimen.

In step (6), the liver of the recipient rat is perfused with the physiological saline through mesenteric vein, then the perihepatic tissues are dissociated to obtain a liver specimen, and the liver specimen is soaked in a 4% neutral paraformaldehyde liquid.

In step (7), corresponding parameters are set on an automatic biochemical analyzer. The plasma is added into the automatic biochemical analyzer, and the automatic biochemical analyzer automatically measures the plasma and outputs results.

### Embodiment 3

(1) A fixed tissue cutting includes the following steps, liver tissues of the recipient are removed from a fixative (also referred to as fixing solution), and the liver tissues are cut with a thickness about 3 millimeters (mm) by using a scalpel in a fume hood to obtain cut liver tissues. The cut liver tissues and corresponding labels thereof are placed in a dehydration box.

(2) A paraffin sections dewaxing to water process includes the following steps; a paraffin section of the liver tissues is sequentially placed in dimethylbenzene I for 20 min, in dimethylbenzene II for 20 min, in anhydrous ethanol I for 5 min, in anhydrous ethanol II for 5 min, and in alcohol with a concentration of 75% for 5 min to obtain a reacted paraffin section, and the reacted paraffin section is washed by tap-water to obtain a washed paraffin section.

(3) A hematoxylin staining process includes the following steps: the washed paraffin section is placed into a hematoxylin stain for 3-5 min to obtain a stained paraffin section, and the stained paraffin section is sequentially washed by the tap-water, differentiate by a differentiation fluid, washed by the tap-water, returned to blue by a bluing reagent, and rinsed by flowing water to obtain a hematoxylin-stained paraffin section.

(4) An eosin staining process includes the following steps: the hematoxylin-stained paraffin section is sequentially placed in alcohol with a concentration of 85% and a concentration of 95% to dehydrate for 5 min to obtain a dehydrated paraffin section, and the dehydrated paraffin section is placed in an eosin stain for 5 min to obtain an eosin-stained paraffin section.

(5) A dehydration process and a sealing process include the following steps: the eosin-stained paraffin section is sequentially placed in the anhydrous ethanol I for 5 min, in the anhydrous ethanol II for 5 min, in anhydrous ethanol III for 5 min, in the dimethylbenzene I for 5 min, in the dimethylbenzene II for 5 min to render them completely transparent to obtain a transparent paraffin section, and the transparent paraffin section is sealed with a neutral balsam to obtain a sealed paraffin section.

(6) The sealed paraffin section is detected by using a microscopic to obtain images, and the images are collected and analyzed.

Analyzed results are as follows.

Nuclei appear blue, and cytoplasm appears red.

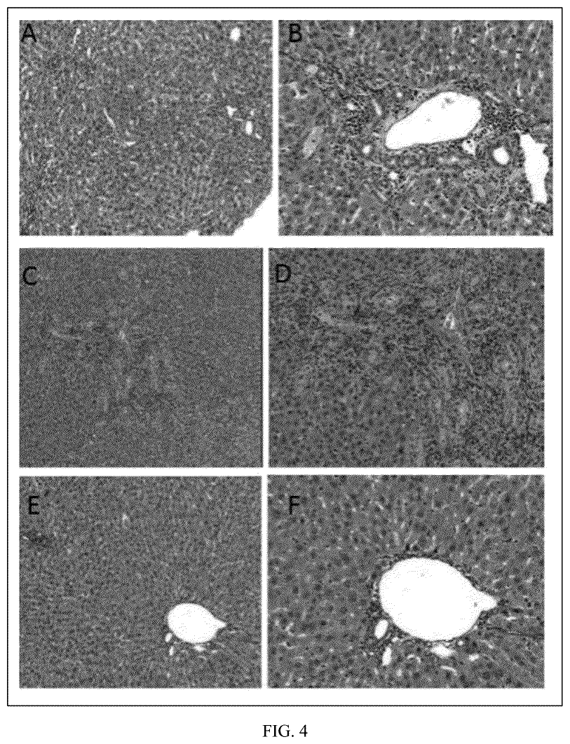

Parts A-B of FIG. 4 illustrate hematoxylin-eosin staining comparison of whole-liver transplants (i.e., the control group 1). A small amount of lymphocytes can be seen infiltrating the liver tissue and most portal areas, and slight interlobular cholangitis can be seen in some portal areas, the lymphocytes can be seen depositing and infiltrating in some hepatic sinuses of hepatic lobules at the same time to indicate that there are very slight or suspected acute rejection; meanwhile, a small amount of pseudobile duct hyperplasia can be observed in some portal areas to indicate that there are very few pseudobile duct hyperplasia manifestations in the portal area caused by ischemic damage factors in the liver tissue. Overall, a degree of the acute rejection is also very slight, when scoring based on the acute rejection of human liver transplantation, that is a rejection activity index (RAI), and RAT is 2, that is, the suspected acute rejection or an uncertain acute rejection reaction.

Parts of C-D of FIG. 4 illustrate hematoxylin-eosin staining comparison of half-liver transplants (i.e., the control group 2). More lymphocytes can be seen infiltrating in the portal areas of the liver tissue, and slight proliferation of fibrous tissue and pseudobile ducts can be seen, and some proliferated fibrous tissues extend to adjacent portal areas and liver tissues to cause slight disorder of local liver tissue structure, but no further pseudolobule formation is found.

Parts of E-F of FIG. 4 illustrate hematoxylin-eosin staining comparison of a tolerance group (i.e., the experimental group). A structure of hepatic lobules is regular, obvious lymphocyte infiltration is not seen in the portal areas, and structure of interlobular bile ducts is present and good, and there is no evidence of edema, necrosis, or cholestasis in liver cells.

What is claimed is:

1. A construction method of an immune tolerance induction scheme for orthotopic liver allograft transplantation, wherein the immune tolerance induction scheme is a scheme to stop an immunosuppressant for life.

2. The construction method as claimed in claim 1, comprising:
removing a liver of a recipient, transplanting a half-liver of a donor for the recipient, providing a model for liver regeneration as a basis to mobilize host bone marrow stem cells; injecting subcutaneously granulocyte colony-stimulating factor (GSF) with a concentration of 150 micrograms per kilogram (μg/kg) to the half-liver 4 hours after operation; injecting subcutaneously the GSF with a concentration of 50-150 μg/kg to the half-liver on 1^{st}, 2^{nd}, 3^{rd}, and 4th days after the operation, once a day, and a total of 5 times; transplanting the half-liver to the recipient and opening blood flow, injecting subcutaneously cyclosporin (CSA) with a concentration of 5-8 milligrams per kilogram (mg/kg) to the half-liver; and injecting subcutaneously the CSA with a concentration of 3-4 mg/kg daily, once a day, and a total of 10 times.

3. An application method of the immune tolerance induction scheme for orthotopic liver transplantation constructed as claimed in claim 2, comprising:
applying the immune tolerance induction scheme for orthotopic liver transplantation to induce immune tolerance in organ transplantation.
